SEQUENCE LISTING (6 sequences)

SEQ ID NO: 1 - protein, 311 aa, Homo sapiens
MRGAARAAWGRAGQPWPRPPAPGPPPPPLPLLLLLLAGLLGGAGAQYSSDRCSWKGSGLTHEAHRKEVEQVYLRCAAGAVEWMYPTGALIVNLRPNTFSPARHLTVCIRSFTDSSGANIYLEKTGELRLLVPDGDGRPGRVQCFGLEQGGLFVEATPQQDIGRRTTGFQYELVRRHRASDLHELSAPCRPCSDTEVLLAVCTSDFAVRGSIQQVTHEPERQDSAIHLRVSRLYRQKSRVFEPVPEGDGHWQGRVRTLLECGVRPGHGDFLFTGHMHFGEARLGCAPRFKDFQRMYRDAQERGLNPCEVGTD

SEQ ID NO: 2 - protein, 311 aa, Rattus rattus
MRGVVWAARRRAGQQWPRSPGPGPGPPPPPPLLLLLLLLLGGASAQYSSDLCSWKGSGLTREAHSKEVEQVYLRCSAGSVEWMYPTGALIVNLRPNTFSPAQNLTVCIKPFRDSSGANIYLEKTGELRLLVRDVRGEPGQVQCFSLEQGGLFVEATPQQDISRRTTGFQYELMSGQRGLDLHVLSAPCRPCSDTEVLLAICTSDFVVRGFIEDVTHVPEQQVSVIHLRVSRLHRQKSRVFQPAPEDSGHWLGHVTTLLQCGVRPGHGEFLFTGHVHFGEAQLGCAPRFSDFQKMYRKAEERGINPCEINME

SEQ ID NO: 3 - protein, 311 aa, Mus musculus
MRGAVWAARRRAGQQWPRSPGPGPGPPPPPPLLLLLLLLLGGASAQYSSDLCSWKGSGLTREARSKEVEQVYLRCSAGSVEWMYPTGALIVNLRPNTFSPAQNLTVCIKPFRDSSGANIYLEKTGELRLLVRDIRGEPGQVQCFSLEQGGLFVEATPQQDISRRTTGFQYELMSGQRGLDLHVLSAPCRPCSDTEVLLAICTSDFVVRGFIEDVTHVPEQQVSVIYLRVNRLHRQKSRVFQPAPEDSGHWLGHVTTLLQCGVRPGHGEFLFTGHVHFGEAQLGCAPRFSDFQRMYRKAEEMGINPCEINME

SEQ ID NO: 4 - DNA, 1348 bp, Homo sapiens
gcggggggcgcgcgacgtgaccacccggactcgaagcccgccccgcccccgcccggctcgccggctccggggtctgctccgggggtcgcggacgcggggccgggcggcggagccggcgccagagcatgcggggcgcggcgcgggcggcctgggggcgcgcggggcagccgtggccgcgaccccccgccccgggcccgcccccgccgccgctcccgctgctgctcctgctcctggccgggctgctgggcggcgcgggcgcgcagtactccagcgaccggtgcagctggaaggggagcgggctgacgcacgaggcacacaggaaggaggtggagcaggtgtatctgcgctgtgcggcgggtgccgtggagtggatgtacccaacaggtgctctcatcgttaacctgcggcccaacaccttctcgcctgcccggcacctgaccgtgtgcatcaggtccttcacggactcctcgggggccaatatttatttggaaaaaactggagaactgagactgctggtaccggacggggacggcaggcccggccgggtgcagtgttttggcctggagcagggcggcctgttcgtggaggccacgccgcagcaggatatcggccggaggaccacaggcttccagtacgagctggttaggaggcacagggcgtcggacctgcacgagctgtctgcgccgtgccgtccctgcagtgacaccgaggtgctcctagccgtctgcaccagcgacttcgccgttcgaggctccatccagcaagttacccacgagcctgagcggcaggactcagccatccacctgcgcgtgagcagactctatcggcagaaaagcagggtcttcgagccggtgcccgagggtgacggccactggcaggggcgcgtcaggacgctgctggagtgtggcgtgcggccggggcatggcgacttcctcttcactggccacatgcacttcggggaggcgcggctcggctgtgccccacgcttcaaggacttccagaggatgtacagggatgcccaggagagggggctgaacccttgtgaggttggcacggactgactccgtgggccgctgcccttcctctcctgatgagtcacaggctgcggtgggcgctgcggtcctggtggggccgtgcggtgagggccgcgcgctgggagccgcatgccctgggcccaggcctgaccctggtaccgaagctgtggacgttctcgccacactcaaccccatgagcttccagccaaggatgccctggccgattggaaatgctgtaaaatgcaaactaagttattatatttttttttggtaaaaaagaaatgtccataggaaacaaaaaaaaaaaaaaaaaa

SEQ ID NO: 5 - DNA, 1380 bp, Rattus rattus
ggcagccggcgcgcttctctggttgcagcttgggcggctggggcggctcctatggtgggcggccaggggctagacgggatggcctgtagacgcgcgacgtgatcagctcgcacgcggacccacgcctcccgcagcactgcctcaacagtctattctgtgggtgcaggcacgcaccggtctcagaccctgccggagcatgcggggtgtggtgtgggcggcccggaggcgcgcggggcagcagtggcctcggtccccgggccctgggccgggtccgcccccgccgccaccgctgctgttgctgctactgctgctgctgggcggcgcgagcgcgcagtactccagcgacctgtgcagctggaaggggagtgggctcacccgggaggcacacagcaaggaggtggagcaggtgtacctgcgctgctcagcaggctctgtggaatggatgtacccaaccggggcgctcattgttaacctacggcccaacaccttctcacctgcccagaacttgactgtgtgcatcaagcctttcagggactcctctggggccaatatttatttggaaaaaactggagaactaagactgttggtgcgggatgtcagaggcgaacctggccaagtgcagtgcttcagcctagagcagggaggcttatttgtggaggccacaccccagcaggacatcagcagaaggaccacaggcttccagtatgagctgatgagtgggcagaggggactggacctgcacgtgctctctgccccctgtcgaccttgcagcgacactgaggtcctccttgccatctgcaccagtgactttgttgtccgaggcttcatcgaggatgtcacccatgtaccagaacagcaagtgtcagtcattcacctacgggtgagcaggctccacaggcagaagagcagggtcttccagccagctcctgaggacagtggccactggctgggccatgtcacaacactgttgcagtgtggagtacgaccagggcatggagaattcctcttcactggacatgtgcactttggggaggcacaacttggatgtgccccacgctttagtgactttcaaaagatgtacaggaaagcagaagaaaggggcataaacccttgtgaaataaatatggagtgacttgcagggtgacaccgtactgctgtccttcagatgagccatggctcagttgctctatcaaatcccgatagagattgcagactggtggcatgagccccgcctggtgcttgaactgggaagggaggtacatgctgctctgaccccttaggtcccattcaaggatgccctgacccattggaaatgttgtaaaatgcaaactaagttattatattttttttgtaaaagaaaaaaaaaaaaaaaaaaaaaaa

SEQ ID NO: 6 - DNA, 2468 bp, Mus musculus
agaggttctaggggcagccggcgcgcttctctagttgcagcttgggcggctcctgtggtgggcggctaggggcgagccgggatgggctatagacgcgcgacgtgatcagttcgcacgcggacccacgcctcccatcgctctgcctcaagagcctattctgtgggtgcaggcacgcaccggacgcagacccggccggagcatgcggggtgcggtgtgggcggcccggaggcgcgcggggcagcagtggcctcggtccccgggccctgggccgggtccgcccccgccgccaccgctgctgttgctgctactactgctgctgggcggcgcgagcgctcagtactccagcgacctgtgcagctggaaggggagtgggctcacccgagaggcacgcagcaaggaggtggagcaggtgtacctgcgctgctccgcaggctctgtggagtggatgtacccaactggggcgctcattgttaacctacggcccaacaccttctcacctgcccagaacttgactgtgtgcatcaagcctttcagggactcctctggagccaatatttatttggaaaaaactggagaactaagactgttggtgcgggacatcagaggtgagcctggccaagtgcagtgcttcagcctggagcagggaggcttatttgtggaggcgacaccccaacaggacatcagcagaaggaccacaggcttccagtatgagctgatgagtgggcagaggggactggacctgcacgtgctgtctgccccctgtcggccttgcagtgacactgaggtcctccttgccatctgtaccagtgactttgttgtccgaggcttcattgaggacgtcacacatgtaccagaacagcaagtgtcagtcatctacctgcgggtgaacaggcttcacaggcagaagagcagggtcttccagccagctcctgaggacagtggccactggctgggccatgtcacaacactgctgcagtgtggagtacgaccagggcatggggaattcctcttcactggacatgtgcactttggggaggcacaacttggatgtgccccacgctttagtgactttcaaaggatgtacaggaaagcagaagaaatgggcataaacccctgtgaaatcaatatggagtgacttgcagggtgacacagtactgttgtccttcagatgagccatgttttgtgggctcagtcgctctatcatatcctgatagagattgcagactggtggcatgggcccagcctggtgctagaactgggaaggtacatgctgctctgaccccttaggtcccagccaaggatgccctgacccattggaactgctgtaaaatgcaaactaagttattatattttttttgtaaaagatgccttggtgtgccatttaatagtgtttttacaaagttattttcaggcattggatttggcctggtatattggtgggagctaggttatggtgtgcagtgatggctatggctcagccttgttattcctgtgatggaaatgtatggagcaaatactttctaatttccccttcattttattttctattttaaaagaccatctttgccgttgagaacctttccagactgtatggaggctgctcccattccagggagtaaagaccaggatctgagactagtattacatccatcttaacccatcagatgggtacctgcattgaaccttctctgctcagctatggcctgctgtcccaaagaccttttgctctctggacagttccagatggtgctgcctggcttaagggacttgttcctcccttgctcctaccaggccactgttgctttctgcatctgtcccactgaaccagtcttgtcctttgaccctgagtttccccaaatgcacacatcaaatccctgaataccaagggactaacctacttaatggcccatttcttcagagggtgtgggttttccctatagtaagaaaatctccacaagttgaagcttaaacagtaggctttcgttcatacagtcctggaagccagaatgggtgtgagcagaatcacatttcctccggagactccaggagggactttatagcttctggtgactccaggaatccttggcttgtaacaatttcactctggcattgctttccctgccatgtgacttctgccttgtatgtgagggcctgtatcaaatctctgtcttgggaggatacagatcattgacttagggcccactccggtgacctcaccttcacctgaaatttactcgatttccatttaggtcagaggcaaaggctacaaaaaatatcaaatccggagaaagattcaatggttaggcacttgctactcttacaaaggacctgtgttcgattcccatgttgggaactcatgttaggtggcttaaaattgcctataactacaattccaggggatctagcaacctcttctcgccacacacaagcacacacacacacacacacacacacacacaattaaaaac

(19) United States
(12) Patent Application Publication
(10) Pub. No.: US 2016/0151459 A1
(43) Pub. Date: Jun. 2, 2016

(54) THERAPEUTIC METHOD OF TREATING METABOLIC DISORDERS

(71) Applicant: EXERKINE CORPORATION, Hamilton (CA)

(72) Inventors: Mark Tarnopolsky, Hamilton (CA); Adeel Safdar, Hamilton (CA)

(21) Appl. No.: 14/943,463

(22) Filed: Nov. 17, 2015

Related U.S. Application Data
(63) Continuation-in-part of application No. PCT/CA2014/000439, filed on May 16, 2014.
(60) Provisional application No. 61/824,702, filed on May 17, 2013.

(51) Int. Cl. A61K 38/22; A61K 45/06

(52) U.S. Cl. CPC A61K 38/22; A61K 45/06

(57) ABSTRACT

A method of treating metabolic disorders in a mammal is provided. The method comprises the step of administering to the mammal a meteorin-like protein or nucleic acid encoding a meteorin-like protein to the mammal.

### FIELD OF THE INVENTION

The present invention relates to a method of treating metabolic disorders, and more particularly, to a method of treating disorders resulting from obesity and obesity-associated co-morbidities including Non-Alcoholic Fatty Liver Disease (NAFLD) and Non-Alcoholic Steato-Hepatitis (NASH).

### BACKGROUND OF THE INVENTION

Obesity and obesity-associated co-morbidities continue to represent major challenges to the health care systems of Canada and other countries. Current therapies are helpful, especially for type 2 diabetes; however, they remain inadequate to prevent the negative effects of obesity on the cardiovascular system, cancer and other aging-associated co-morbidities. It has long been recognized that exercise is an excellent first line therapy for both type 2 diabetes and obesity; however, many patients, especially morbidly obese, are unable to exercise sufficiently for a variety of reasons. Exercise is also a very effective therapy for older adults to increase muscle mass and strength and to lower the risk of many age-associated disorders including; cancer, cardiovascular disease and cognitive impairment. Finally, long-term participation in vigorous exercise is associated with a lower risk of all causes of mortality, cardiovascular disease, stroke, osteoporosis, cancer risk and incidence of neurological disorders. A huge challenge has been to “capture” some of the benefits of exercise in a manner that can be useful medically for the very broad range of diseases for which exercise appears to provide benefit.

Endurance exercise induces metabolic adaptations via activation of the transcriptional co-activator, peroxisome proliferator-activated receptor γ coactivator-1α (PGC-1α). PGC-1α is the master regulator of mitochondrial metabolism and biogenesis, and has been touted as a potential therapeutic target for obesity and obesity-associated co-morbidities, including diabetes and Non-Alcoholic Fatty Liver Disease (NAFLD) and Non-Alcoholic Steato-Hepatitis (NASH). Interestingly, mild over-expression of PGC-1α in skeletal muscle alone is known to be protective against sarcopenia, to attenuate inactivity-induced fiber atrophy, to ameliorate ALS pathology, to reduce systemic chronic inflammation, and to maintain systemic glucose and insulin homeostasis in aged mice.

Accordingly, it would be desirable to further understand the metabolic effects of exercise in order that new therapies may be developed based on the benefits of exercise.

### SUMMARY OF THE INVENTION

It has now been determined that the protein, meteorin-like protein (METRNL), stimulates “browning” of subcutaneous white adipose fat depot, and thus, is useful in the treatment of metabolic disorders including obesity and obesity-associated co-morbidities, such as, but not limited to; obesity, type 2 diabetes, insulin resistance, NAFLD and NASH.

Thus, in one aspect of the invention, a method of treating obesity and obesity-associated co-morbidities in a mammal is provided comprising the administration of meteorin-like protein or nucleic acid encoding meteorin-like protein to the mammal.

In another aspect of the invention, a composition for the treatment of obesity and obesity-associated co-morbidities is provided comprising meteorin-like protein or nucleic acid encoding meteorin-like protein in combination with a pharmaceutically acceptable carrier.

These and other aspects of the invention will be described by reference to the following figures.

### BRIEF DESCRIPTION OF THE FIGURES

FIG. 1 graphically illustrates that muscle-specific PGC1-α transgenic mice have increased brown/beige-like fat cells in the subcutaneous fat depot as shown by: (A) Quantitative polymerase chain reaction (qPCR) for brown fat and thermogenic genes in subcutaneous fat depot in muscle creatine kinase promoter (MCK)-PGC1-α transgenic mice or littermate wild-type (WT) control mice (n=6/group), (B) Western blot against UCP1 (a marker of brown/beige fat cells) in subcutaneous fat depot from MCK-PGC1-α (MP) and littermate control (WT) mice (n=6/group), (C) qPCR against indicated genes in adipocytes differentiated for 6 days from stromo vascular fraction (SVF) cells, and (D) an increase in Ucp1 mRNA expression in subcutaneous inguinal fat depot following 4 weeks of forced treadmill exercise. Data are presented as Mean±S.E.M., and *P<0.05 compared to control group. Student's t-test was used for statistical analyses.

FIG. 2 graphically illustrates that METRNL is induced with transgenic PGC1-α over-expression or endurance exercise, and activates brown/beige fat gene expression programming by: (A) qPCR for indicated genes in skeletal muscle from MCK-PGC1-α transgenic mice or littermate controls (n=6/group), (B) qPCR for indicated genes in skeletal muscle from sedentary mice or mice underwent acute bout of forced treadmill exercise (15 m/min for 90 min; n=6/group) followed by three hours of recovery, (C) qPCR for indicated genes in skeletal muscle from muscle-specific PGC1-α knockout mice or littermate flox/flox controls (n=6/group), (D) mRNA expression levels from human muscle biopsies before and after three hours of an acute exhaustive bout of endurance exercise (˜75 min graded interval workout to exhaustion on a cycle ergometer; n=9), and (E) mRNA expression in SVF from the subcutaneous fat depot, differentiated into adipocytes for 6 days in the presence of PBS (vehicle control) or recombinant METRNL (20 nM). For (E), one-way ANOVA statistical test was performed where *P<0.05. All other statistics were performed using Student's t-test, and bar graphs are Mean±S.E.M.

FIG. 3 graphically illustrates the effect of anti-METRNL neutralizing antibodies on the ability of the PGC-1α conditioned media to increase the browning gene expression in primary inguinal cells (A), and the effect of VEGFA, VEGFB, and PF4 on Ucp1 expression (B).

FIG. 4 graphically illustrates that METRNL is a potent inducer of the brown/beige fat gene expression programming in mice and human fat cells by: (A) mRNA levels of Ucp1 from subcutaneous-derived SVF, differentiated into adipocytes, treated with METRNL for 6 days at indicated doses during differentiation, (B) Subcutaneous explants from type 2 diabetes patients (n=3) were treated in an ex vivo adipose tissue culture with PBS (vehicle) or METRNL (20 nM) for 4 days, following which mRNA was isolated and analyzed using qPCR for browning gene program, (C) Seahorse XFe Analyzer measurements of basal and uncoupled oxygen consumption rate (nmol/min) in SVF from the subcutaneous fat depot, differentiated into adipocytes for 6 days in the presence of PBS or recombinant METRNL (20 nM), and (D) qPCR of Ppara from subcutaneous-derived SVF, differentiated into adipocytes, treated with METRNL (20 nM) for 6 days. All statistics were performed using Student's t-test, where *, †P<0.05 and bar graphs are Mean±S.E.M.

FIG. 5 graphically illustrates that ex vivo treatment of human subcutaneous fat pads with METRNL induced a browning gene program and mitochondrial oxidative metabolism gene expression (A), and that METRNL was effective to induce Ucp1 mRNA throughout the differentiation process of pre-adipocytes (B).

FIG. 6 graphically illustrates that effect of exercise in mice (A) and humans (B) on blood levels of METRNL.

FIG. 7 graphically illustrates the effect of exogenous METRNL on Ucp1 expression in the subcutaneous fat depot in mice (A), and on UCP1 protein in inguinal fat (B).

FIG. 8 illustrates that METRNL induces browning of white adipose tissue in vivo and protects against diet-induced obesity, diabetes and non-alcoholic liver disease (NAFLD, NASH). C57BL/6 mice fed a 60% kcal high-fat diet for 20 weeks were then subjected to forced-treadmill running (END exercise group [gold standard]; 15 m/min for 60 min; 3× week for 2 months) (n=8 per group) or were injected with saline (control group) or recombinant mouse METRNL (experimental group; 0.4 ng/kg METRNL) intravenously for either 30 consecutive days (FIG. 8A-F) or 3 times per week for 7 weeks (FIG. 8G-I). (A) Subcutaneous fat depots were collected and analyzed using qPCR for browning gene program and mitochondrial genes. (B) Oxygen consumption at day and night. (C) Body weight of mice after 30 days of treatment. (D) Fasting plasma insulin measured using ELISA. (E/F) Glucose tolerance test of mice after 30 days of treatment and area under the curve. (G) Representative images of Oil red O staining in liver and (H) corresponding quantification of total hepatic lipid infiltration. (I) Scoring of hepatic steatosis. One-way ANOVA was used for statistics, where *P<0.05 (vs. Saline) and dagger P<0.05 (vs. METRNL). Bar graphs are Mean±S.E.M.

FIG. 9 illustrates that exercise induced browning of white adipose tissue in vivo is in part mediated by METRNL. Mice were injected intraperitoneally with 50 μg of sheep IgG or a sheep anti-METRNL antibody and were either subjected to forced-treadmill exercise for 30 days or kept sedentary (n=10 for all groups). Data show mRNA expression levels from inguinal white adipose tissue. One-way ANOVA was used for statistics, where *P<0.05 (vs. Saline) and dagger P<0.05 (vs. METRNL). Bar graphs are Mean±S.E.M.

FIG. 10 illustrates the amino acid sequences of human meteorin-like protein (A), rat (B) and mouse (C), a sequence alignment between mouse and human sequences (D) and similarity between mouse METRNL vs. other species (E).

FIG. 11 illustrates the METRNL-encoding nucleic acid sequences in human (A), rat (B) and mouse (C).

### DETAILED DESCRIPTION OF THE INVENTION

A method of treating metabolic disorders in a mammal is provided comprising the step of administering meteorin-like protein or nucleic acid encoding meteorin-like protein to the mammal.

The term “metabolic disorder” is used herein to encompass disorders resulting from obesity and obesity-associated co-morbidities, including but not limited to, obesity, type 2 diabetes, dysglycemia, insulin resistance, Non-Alcoholic Steato-Hepatitis (NASH) and Non-Alcoholic Fatty Liver Disease (NAFLD).

The term “obesity” is used herein to describe a condition of excess adipose tissue and to encompass individuals classified as at least overweight with a body mass index greater than or equal to 25 kg/m², and preferably, individuals classified as obese having a body mass index greater than or equal to 30 kg/m².

The term “obesity-associated co-morbidities” is used herein to encompass any disease, condition or disorder which is known to occur with higher incidence or prevalence in obese individuals, or known to be caused at least in part by obesity and is exemplified, but not limited to, type 2 diabetes, dysglycemia, insulin resistance, Non-Alcoholic Steato-Hepatitis (NASH) and Non-Alcoholic Fatty Liver Disease (NAFLD).

The term “browning” is used herein to encompass a change in white adipose tissue in which it expresses features that are characteristic of the brown adipose tissue program exemplified, but not limited to, an increased expression of Ucp1, Prdm16 and/or Cidea, an increased uncoupling of cellular respiration, and/or an increased expression of markers of mitochondrial oxidative metabolism such as Cox4il, Cox5a, Cox7a1, Cox8b, Cytc, Ndufb5, Cox-II, Cox-IV, and ATPase 6.

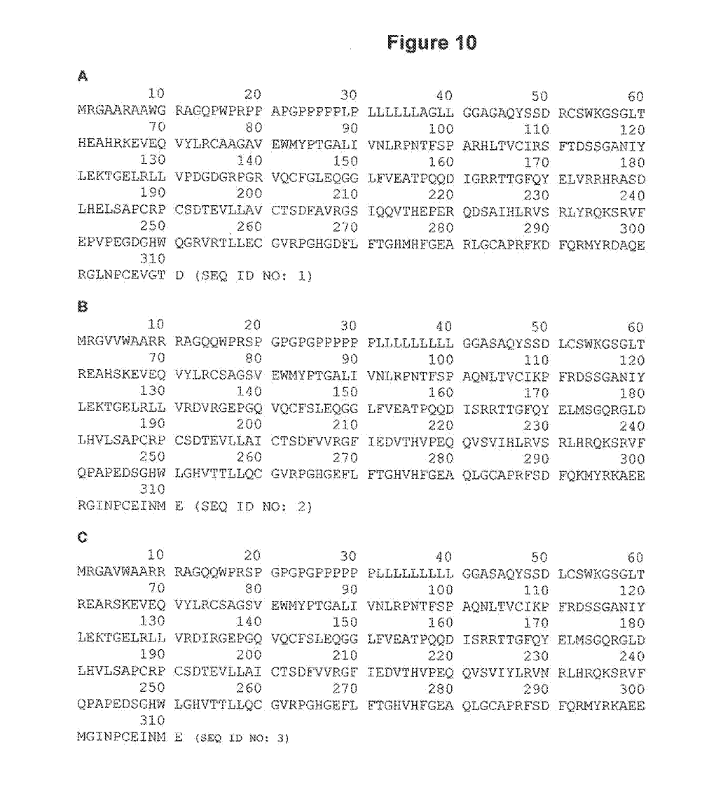

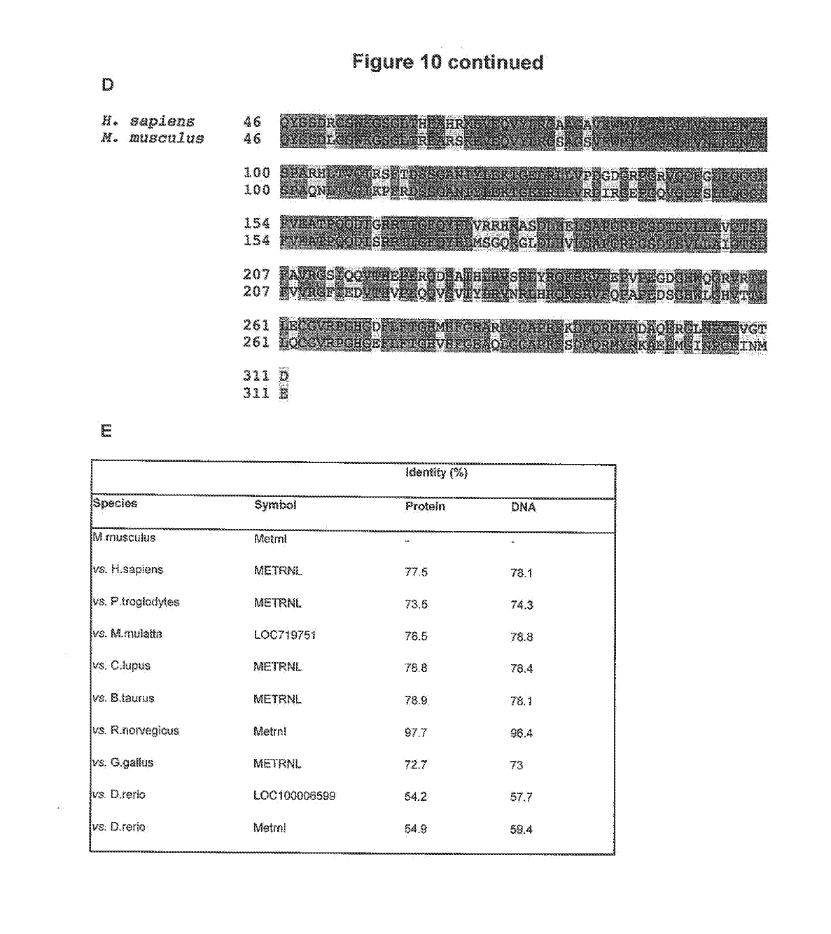

The term “meteorin-like protein” or “METRNL”, also known as glial cell differentiation regulator-like protein, is used herein to encompass mammalian meteorin-like protein (e.g. the wildtype isoforms), including human and non-human meteorin-like protein, and functionally equivalent variants thereof. Human meteorin-like protein is an 811 amino acid protein as shown in FIG. 10A, and examples of functionally equivalent variants thereof include, isoforms thereof and non-human forms, for example, as set out in FIG. 10B/C.

The term “functional equivalent variants” as they relate to meteorin-like protein include naturally or non-naturally occurring variants of an endogenous meteorin-like protein that retain the biological activity of meteorin-like protein, e.g. to treat metabolic syndrome, for example, by increasing expression of UCP1 and other genes of the brown fat program in white adipose cells. The variant need not exhibit identical activity to endogenous meteorin-like protein, but will exhibit sufficient activity to render it useful to treat a metabolic disorder, e.g. at least about 25% of the biological activity of meteorin-like protein, and preferably at least about 50% or greater of the biological activity of meteorin-like protein. Such functionally equivalent variants may result naturally from alternative splicing during transcription or from genetic coding differences and may retain significant sequence similarity with wild-type meteorin-like protein, e.g. at least about 70% sequence similarity, preferably at least about 80% sequence similarity, and more preferably at least about 90% or greater sequence similarity. Such variants can readily be identified using established cloning techniques employing primers derived from meteorin-like protein. Additionally, such modifications may result from non-naturally occurring synthetic alterations made to meteorin-like protein to render functionally equivalent variants which may have more desirable characteristics for use in a therapeutic sense, for example, increased activity or stability. Non-naturally occurring variants of meteorin-like protein include analogues, fragments and derivatives thereof.

A functionally equivalent analogue of meteorin-like protein in accordance with the present invention may incorporate one or more amino acid substitutions, additions or deletions. Amino acid additions or deletions include both terminal and internal additions or deletions to yield a functionally equivalent peptide. Examples of suitable amino acid additions or deletions include those incurred at positions within the protein that are not closely linked to activity. Amino acid substitutions within the meteorin-like protein, particularly conservative amino acid substitutions, may also generate functionally equivalent analogues thereof. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent fragment in accordance with the present invention comprises a portion of a meteorin-like protein sequence which maintains the function of intact meteorin-like protein, e.g. with respect to treating metabolic syndrome, for example by inducing browning of adipose tissue. Such biologically active fragments of a meteorin-like protein can readily be identified using assays useful to evaluate the activity of selected meteorin-like fragments.

A functionally equivalent derivative of meteorin-like protein in accordance with the present invention is meteorin-like protein, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may render a peptide having more desirable characteristics such as increased stability or activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

Meteorin-like protein, and functionally equivalent variants thereof, may be made using standard, well-established solid-phase peptide synthesis methods (SPPS). Two methods of solid phase peptide synthesis include the BOC and FMOC methods. Meteorin-like protein and variants thereof may also be made using any one of a number of suitable techniques based on recombinant technology. It will be appreciated that such techniques are well-established by those skilled in the art, and involve the expression of nucleic acid encoding meteorin-like protein in a genetically engineered host cell. DNA encoding a meteorin-like protein may be synthesized de novo by automated techniques well-known in the art given that the protein and nucleic acid sequences are known.

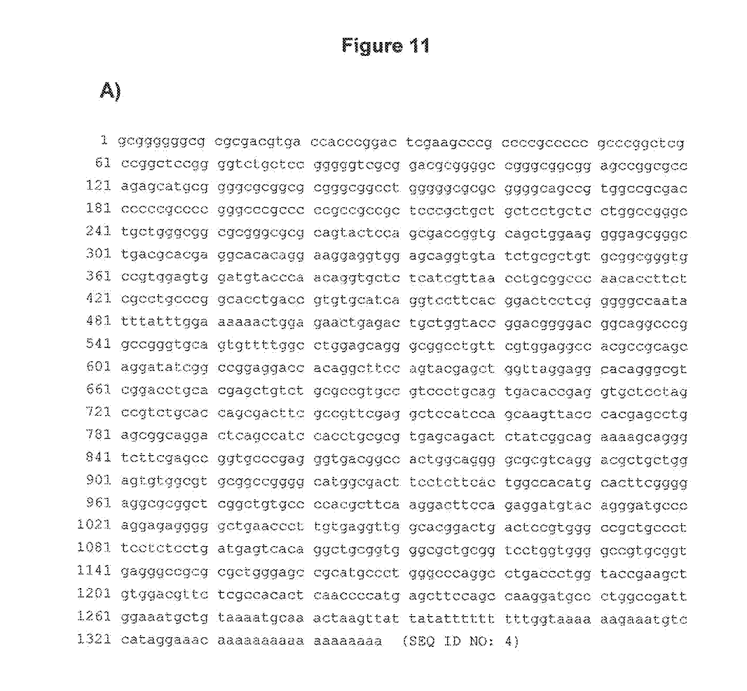

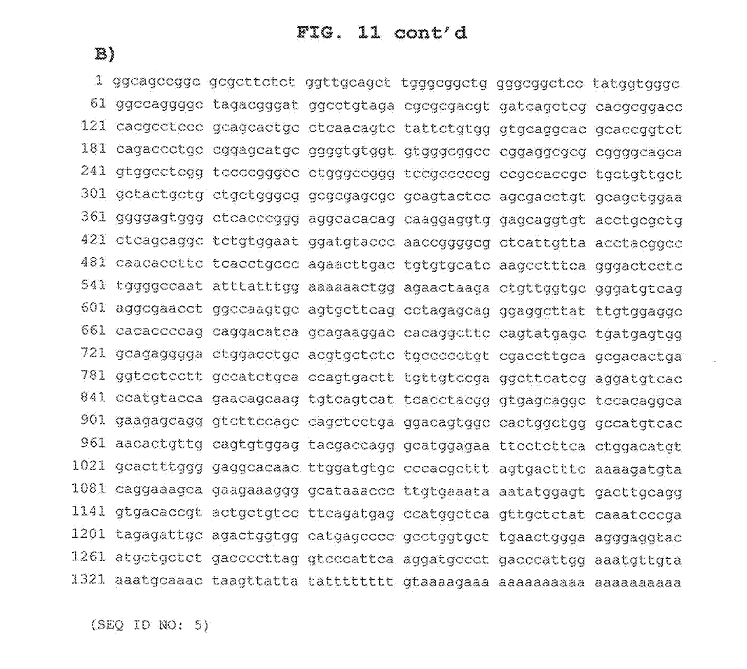

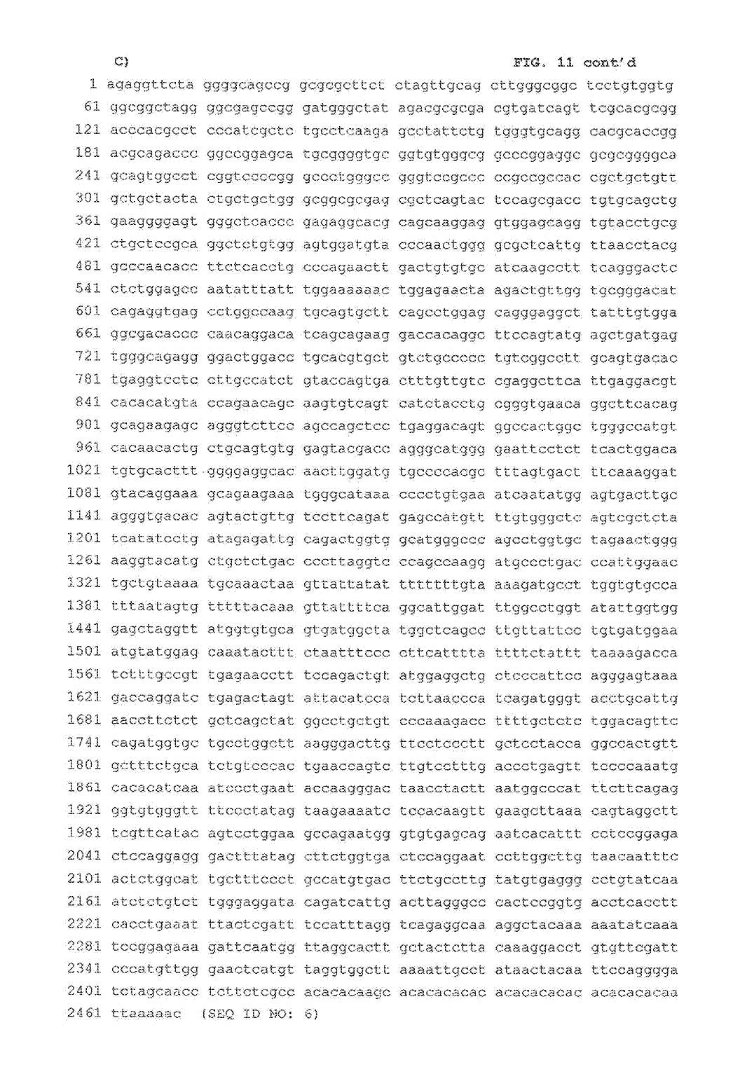

Nucleic acid molecules or oligonucleotides encoding meteorin-like protein (e.g. DNA, mRNA) may also be used to increase plasma meteorin-like protein levels. In this regard, “nucleic acid encoding meteorin-like protein” is used herein to encompass mammalian nucleic acid encoding meteorin-like protein, including human and non-human forms, and functionally equivalent variants thereof (e.g. nucleic acids that encode functionally equivalent meteorin-like protein, or nucleic acids which differ due to degeneracy of the genetic code). The sequence of the human gene encoding meteorin-like protein is shown in FIG. 11A, and examples of functionally equivalent variants, e.g. oligonucleotides encoding non-human METRNL, are shown in FIG. 11B/C, and may additionally be readily accessed, for example, via GenBank and other sequence depositories as is known to those in the art. Functionally equivalent METRNL oligonucleotide variants encode a functionally equivalent variant of human METRNL that exhibits at least about 70% sequence similarity thereto.

The term “oligonucleotide” refers to an oligomer or polymer of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phophorodithioates. Oligonucleotides of the invention may also comprise nucleotide analogs such as peptide nucleic acid (PNA) in which the deoxribose (or ribose) phosphate backbone in the DNA (or RNA), is replaced with a polymide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

Once prepared and suitably purified, meteorin-like protein, oligonucleotides encoding meteorin-like protein, or functionally equivalent variants thereof, may be utilized in accordance with the invention to treat metabolic disorder. In this regard, increasing the expression of meteorin-like protein in a mammal, by administration of a meteorin-like protein or by administration of nucleic acid encoding meteorin-like protein, results in meteorin-like protein expression or over-expression in the mammal. While not wishing to be bound by any particular mode of action, upregulation of meteorin-like protein results in upregulation of metabolites which induce the brown fat program in white adipose cells.

Meteorin-like protein or nucleic acid may be administered either alone or in combination with at least one pharmaceutically acceptable adjuvant, in the treatment of metabolic disorders in accordance with an embodiment of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants are those used conventionally with peptide- or nucleic acid-based drugs, such as diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragacanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water, isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered, for example, anti-microbial agents may be added to the composition to prevent microbial growth over prolonged storage periods.

To increase energy expenditure in the treatment of metabolic disorders, a therapeutically effective amount of a meteorin-like protein or nucleic acid encoding meteorin-like protein is administered to a mammal. As used herein, the term “mammal” is meant to encompass, without limitation, humans, domestic animals such as dogs, cats, horses, cattle, swine, sheep, goats and the like, as well as non-domesticated animals. The term “therapeutically effective amount” is an amount of the meteorin-like protein or nucleic acid encoding meteorin-like protein required to increase or upregulate mitochondrial biogenesis from its existing status, for example, by inducing at least one gene of the thermogenic brown fat program in white adipose cells such as UCP1, Prdm16, Cidea and the like (to increase existing levels thereof), while not exceeding an amount which may cause significant adverse effects. Dosages of meteorin-like protein, functionally equivalent variants thereof, or nucleic acid encoding meteorin-like protein or functionally equivalent variants, that are therapeutically effective will vary with many factors including the nature of the condition to be treated as well as the particular individual being treated. Appropriate dosages of meteorin-like protein or nucleic acid encoding meteorin-like protein for use include dosages sufficient to effect at least about a 10% increase in endogenous plasma levels of meteorin-like protein. In one embodiment, dosages within the range of about 0.1 pg/kg to 4 ng/kg meteorin-like protein are appropriate while dosages of nucleic acid encoding meteorin-like protein that yields or expresses about 0.1 pg/kg to 4 ng/kg are appropriate. The dosage may be delivered on a daily basis or less frequently, e.g. 2, 3, 4, 5 or 6 times per week. In another embodiment, dosages of meteorin-like protein or nucleic acid encoding meteorin-like protein that mimic the results of an exercise regimen are used, e.g. a pulsatile dosage in an amount which increases plasma METRNL levels by at least about 10% of resting endogenous levels, e.g. a dosage of about 0.1 pg/kg to 4 ng/kg meteorin-like protein or a dosage of nucleic acid encoding meteorin-like protein that expresses about 0.1 pg/kg to 4 ng/kg protein 3-5 times per week.

In the present treatment, meteorin-like protein or nucleic acid per se may be administered by any route suitable to increase the plasma levels thereof. Examples of suitable administrable routes include, but are not limited to; oral, subcutaneous, intravenous, intraperitoneal, intranasal, enteral, topical, sublingual, intramuscular, intra-arterial, intramedullary, intrathecal, inhalation, ocular, transdermal, vaginal or rectal means. Depending on the route of administration, the protein or nucleic acid may be coated or encased in a protective material to prevent undesirable degradation thereof by enzymes, acids or by other conditions that may affect the therapeutic activity thereof.

As one of skill in the art will appreciate, meteorin-like protein or nucleic acid may be administered to a mammal in conjunction with a second therapeutic agent to facilitate treatment of the mammal. The second therapeutic agent may be administered simultaneously with the meteorin-like protein or nucleic acid, either in combination or separately. Alternatively, the second therapeutic agent may be administered prior or subsequent to the administration of the meteorin-like protein or nucleic acid. In one embodiment, the second therapeutic agent is an agent that is also useful to treat metabolic disorders, i.e. disorders resulting from obesity and obesity-associated co-morbidities as previously described. Examples of such therapeutic agents include, but are not limited to, one or more of a cytokine such as IL-6, insulin, betatrophin, adiponectin, leptin and a nutritional supplement, including vitamins, minerals and the like, such as alpha lipoid acid, coenzyme Q10, creatine, vitamin E and the like.

In another aspect of the present invention, an article of manufacture is provided. The article of manufacture comprises packaging material and a composition comprising a pharmaceutically acceptable adjuvant and a therapeutically effective amount of meteorin-like protein or functionally equivalent variant thereof. The packaging material is labeled to indicate that the composition is useful to treat metabolic syndrome.

The packaging material may be any suitable material generally used to package pharmaceutical agents including, for example, glass, plastic, foil and cardboard.

Embodiments of the invention are described in the following specific examples which are not to be construed as limiting.

### Example 1

### UCP-1α and Thermogenesis

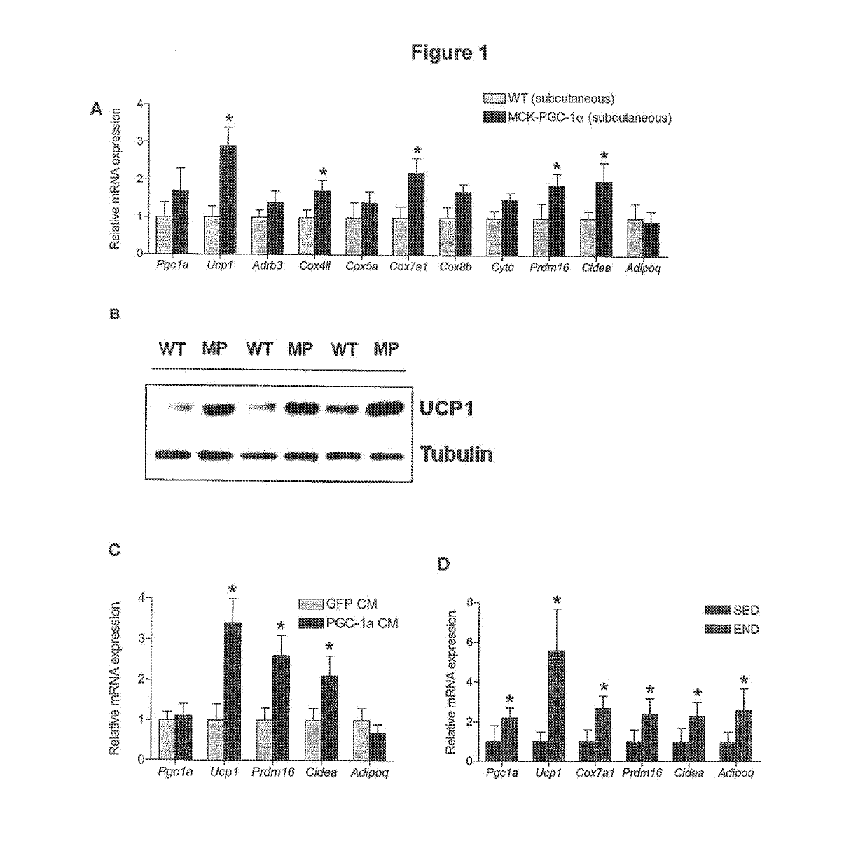

Since mice with transgenic over-expression of PGC-1α in skeletal muscle are resistant to age-related obesity and diabetes, the adipose tissue of PGC-1α transgenic mice was analyzed for expression of genes related to a thermogenic gene program and genes characteristic of brown fat development. There were no significant alterations in the expression of browning genes in the inter-scapular brown adipose tissue or in the visceral (epididymal) white adipose tissue. However, the subcutaneous fat layer (inguinal), a white adipose tissue that is particularly prone to browning (that is, formation of multi-locular, UCP1-positive adipocytes), had significantly higher Ucp1 and Cidea gene expression (FIG. 1A). Higher UCP1 protein levels and more UCP1-positive stained multi-locular cells were also observed in subcutaneous fat depot of PGC-1α transgenic mice compared to litter-mate control mice (FIG. 1B). Since the subcutaneous white adipose depot has the greatest tendency to turn on a thermogenic gene program and alter the systemic energy balance of mice, this phenomenon with regard to browning of the white adipose tissue was investigated with an endurance exercise paradigm. A significant increase in Ucp1 mRNA expression was observed in subcutaneous inguinal fat depot following 4 weeks of forced treadmill running (FIG. 1D).

To explain the effect on browning of the adipose tissues from transgenic muscle over-expressing PGC-1α mice, cultured primary subcutaneous adipocytes were treated with serum-free media conditioned from myotubes expressing PGC-1α vs. myotubes expressing green fluorescent protein (GFP). The conditioned media from cells expressing ectopic PGC-1α increased the gene expression of several browning genes (FIG. 1C). This induction of browning genes is neutralized by prior heat-inactivation or trypsinization of the conditioned media from cells expressing PGC-1α. Together, this suggests that PGC-1α causes the muscle cells to secrete a peptide(s) that can induce a thermogenic gene program in fat cells.

### Example 2

### METRNL and the Thermogenic Program

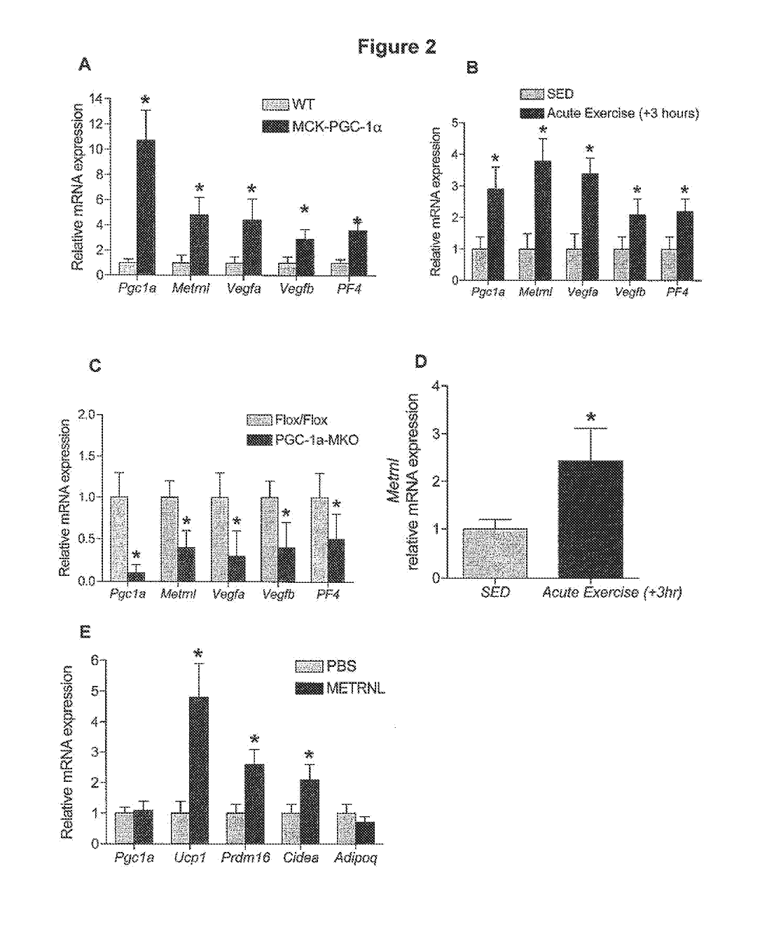

A combination of Illumina-based gene expression arrays and a protein bioinformatics-based algorithm that predicts exerkine secretion were used to search for protein(s) that could mediate the browning of adipose tissues under the control of muscle PGC-1α and with endurance exercise. Proteins with mitochondrial targeting sequences were excluded, and all candidates were validated using an in vivo PGC-1α gain-of-function system (FIG. 2A). Four proteins were identified as PGC-1α target genes (which are also responsive to endurance exercise) in muscle and as likely to be secreted: Metrnl (meteorin-like protein, glial cell differentiation regulator-like), Vegfa (vascular endothelial growth factor A), Vegfb (vascular endothelial growth factor B), and PF4 (platelet factor 4). Conversely, it was found that expression of these genes was reduced in muscle of sedentary mice vs. exercise mice (FIG. 2B) and also in mice with muscle-specific deletion of PGC-1α vs. littermate control mice (FIG. 2C). The expression of this same set of genes was also examined in muscle biopsies from human subjects before and after an acute bout of exercise and after 3 and 6 months of endurance training (FIG. 2D). Metrnl, Vegfa, Vegfb, and PF4 expression were significantly induced in humans with endurance exercise.

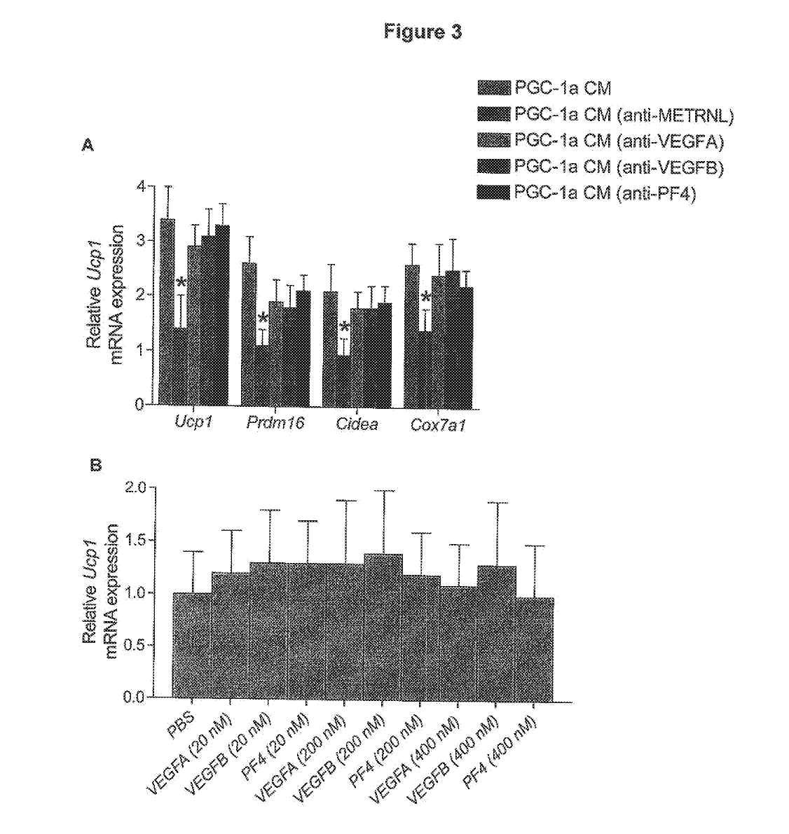

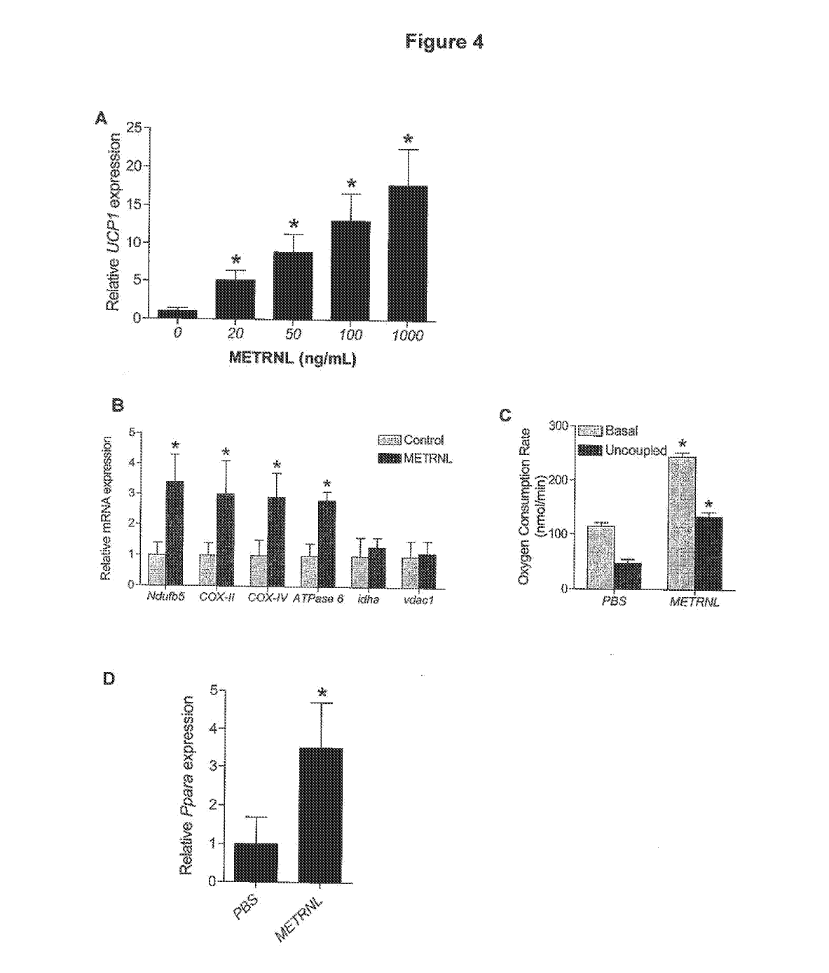

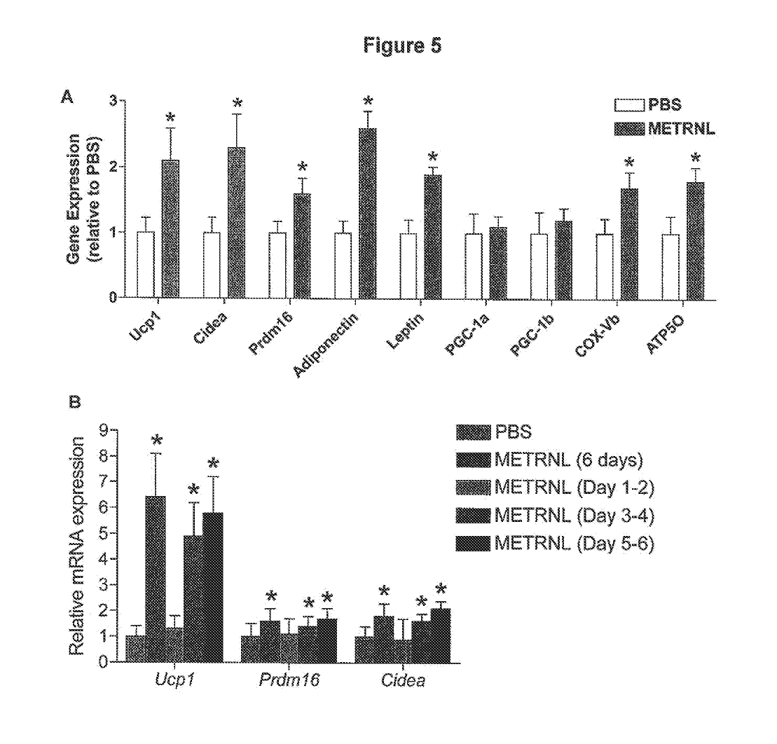

To identify which of the aforementioned predicted genes encoded proteins that act as a browning exerkine(s), conditioned media from cells expressing PGC-1α was treated with neutralizing antibodies before treating primary subcutaneous adipocytes. Conditioned media treated with anti-VEGFA, anti-VEGFB, and anti-PF4 did not ablate the increase in Ucp1 and other browning genes expression. In contrast, anti-METRNL neutralizing antibodies caused a marked reduction in the ability of the PGC-la conditioned media to increase the browning gene expression in primary inguinal cells vs. the control antibody (FIG. 3A). Additionally, the predicted proteins are commercially available, so they were applied directly to the primary subcutaneous white adipocytes during differentiation. Exerkines such as VEGFA, VEGFB, and PF4 had minimal effects on Ucp1 expression at concentrations of 200 nM or higher (FIG. 2E and FIG. 3B). However, recombinant METRNL significantly induced the expression of Ucp1 and other known brown fat genes (Prdm16 and Cidea) and mitochondrial oxidative metabolism genes, and down-regulated the expression of genes characteristic of white fat development at a concentration of 20 nM (FIG. 2E and FIGS. 4, A and B). Similarly, ex vivo treatment of human subcutaneous fat pads with METRNL induced a browning gene program and mitochondrial oxidative metabolism gene expression (FIG. 5A). Lastly, measurements of oxygen consumption provided functional evidence of increased energy expenditure with METRNL exposure in vitro (FIG. 4D). Total oxygen consumption was greatly increased by 20 nM of METRNL, and the majority of this respiration was uncoupled—characteristic of brown fat-like phenotype. These data indicate that the activation of browning and thermogenic genes by METRNL is a major part of the action of this polypeptide on subcutaneous adipocytes.

### Example 3

### METRNL and Cell Differentiation

The time-frame of METRNL-mediated induction of Ucp1 mRNA during the differentiation of pre-adipocytes was then determined. METRNL was applied to cells in 2-day windows from day 0-6, and this was compared to cells to which the protein was added during the entire 6-day differentiation process. Treatment during days 3-6 was effective at inducing Ucp1 mRNA, although not as effective as when METRNL was present throughout the differentiation process (FIG. 5B). Furthermore, treatment during the initial 2 days had no effect on UCP1 levels, suggesting that METRNL acts mainly during the differentiation process of cells committed to the adipocyte lineage.

### Example 4

### Effect of Exogenous METRNL

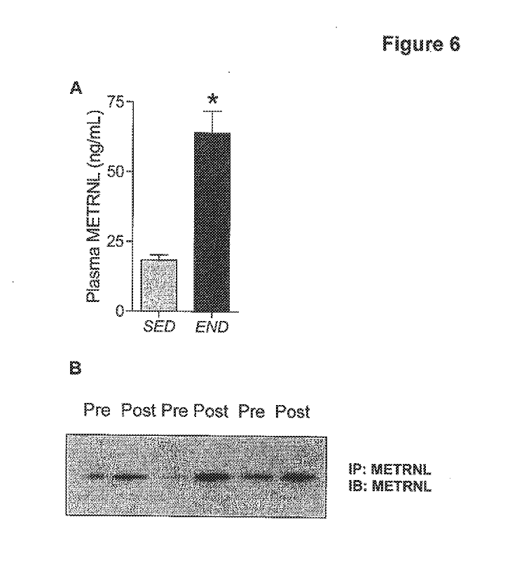

Blood levels of METRNL were determined after exercise in mice and human subjects. Mice had significantly elevated (3.6-fold) plasma concentrations of METRNL after 4 weeks of forced-treadmill endurance exercise (FIG. 6A). Similar analyses in healthy adult humans subjected to supervised endurance exercise training for 12 weeks revealed a significant increase in the circulating METRNL levels vs. pre-exercise plasma (FIG. 6B). The increase in circulating protein in both species corresponds to the increases observed at the mRNA level in muscle.

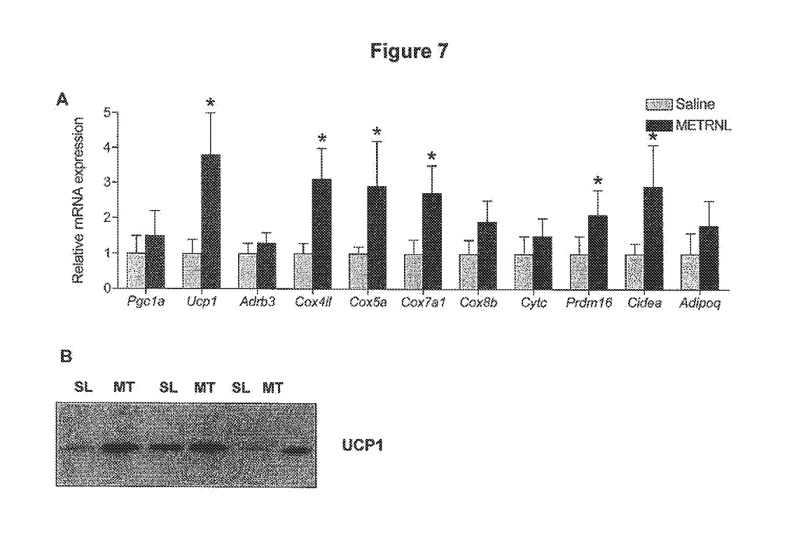

Since endurance exercise is known to cause browning in vivo, it was determined whether or not exogenous recombinant METRNL could recapitulate some the browning aspect of exercise. To test this, recombinant METRNL (0.4 ng/kg) was injected into young healthy C57Bl/6J mice. After 21 days of METRNL injections (3 times weekly), Ucp1 expression was found to be increased by 4-fold in the subcutaneous fat depot relative to the same depot in mice receiving saline injection (FIG. 7A). The changes in gene expression in the subcutaneous adipose tissues were accompanied by a clear increase in the UCP1 protein in inguinal fat (FIG. 7B). This shows that moderate increases in circulating METRNL can induce browning of white adipose tissues in vivo, including increased expression of UCP1.

### Example 5

### METRNL, Glucose Tolerance and Liver Disease

Since exercise-mediated browning of subcutaneous white adipose tissue is intimately linked with improvements in glucose sensitivity and whole-body adiposity and since METRNL is herein shown to induce browning, the effect of METRNL treatment in reversing glucose intolerance and metabolic disorders was determined. To elucidate the therapeutic nature of METRNL, recombinant METRNL (0.4 ng/kg) was injected into a high-fat fed (HFD) mouse model of obesity and type 2 diabetes, either 3 times per week or daily for 28 or 30 days, respectively. C57BL/6 mice were chosen for these experiments because they are prone to diet-induced obesity and diabetes.

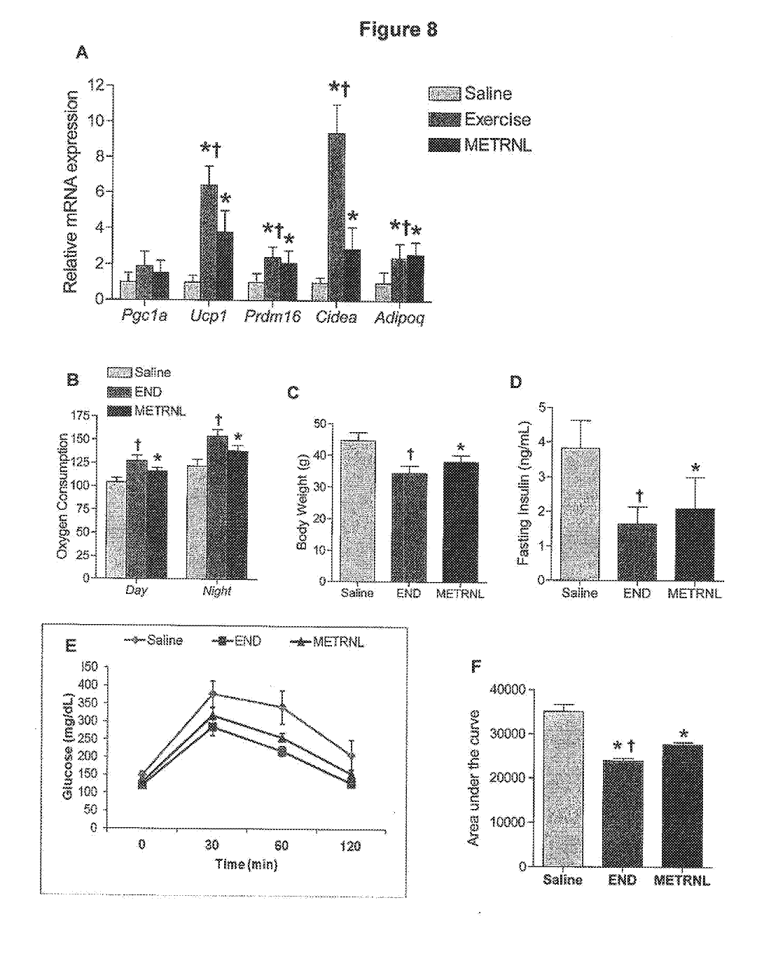

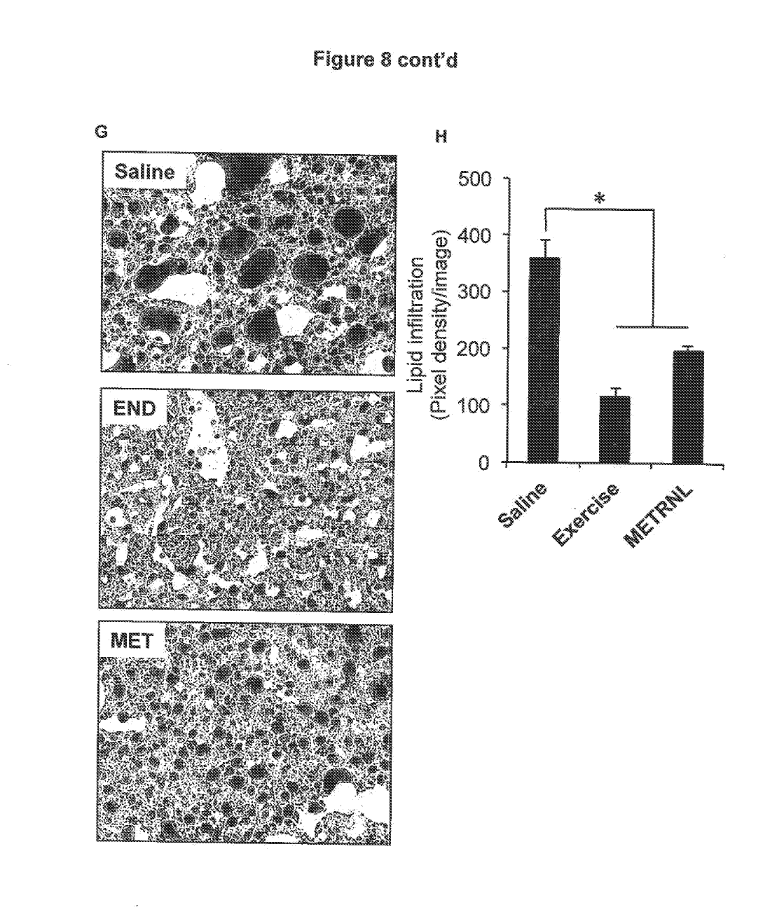

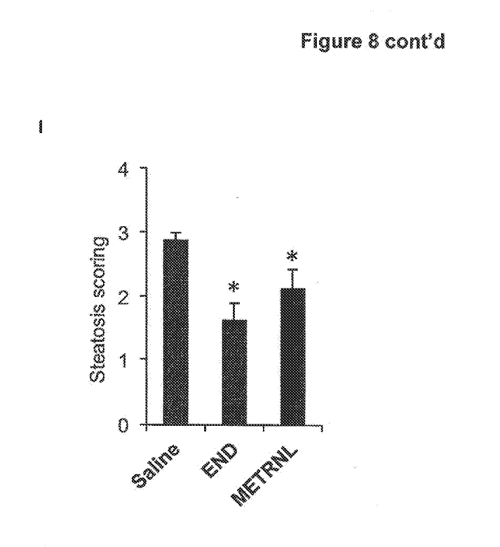

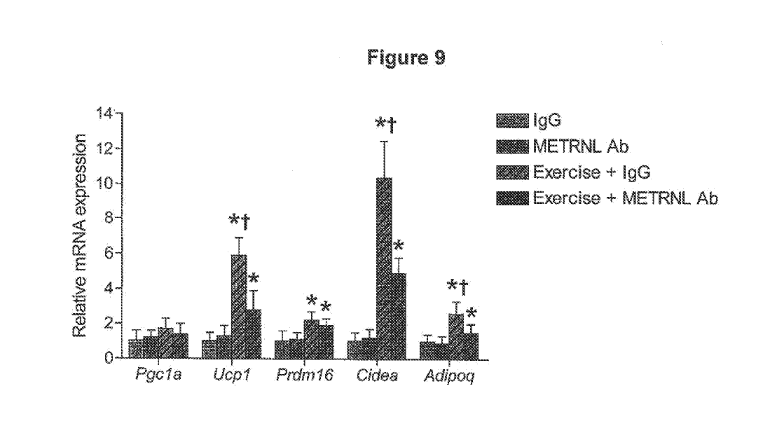

Recombinant METRNL was found to increase Ucp1 gene expression in the inguinal fat from HFD mice to the same degree as in lean mice or HFD mice subjected to endurance exercise (gold-standard for type 2 diabetes therapy) (FIG. 8A). There was also an elevation in expression of several mitochondrial genes similar to exercised HFD mice (FIG. 8A). This effect was accompanied with a large increase in oxygen consumption (FIG. 8B), consistent with the gene expression data. The body weights of the METRNL-injected HFD mice were slightly lower as compared to saline-injected HFD controls (FIG. 8C). Like exercised HFD mice, METRNL-injected HFD mice showed a significant improvement in glucose tolerance and reduction in fasting glucose (FIGS. 8E and F), and fasting insulin (FIG. 8D) compared to the control group. Similarly, both METRNL-injected HFD and exercised HFD mice had less hepatic lipid accumulation or steatosis in the liver (FIG. 8G-I) in comparison to saline-injected HFD controls. These data illustrate that even moderately increased levels of circulating METRNL potently increased energy expenditure, reduced body weight and protected against diet-induced insulin resistance, Non-Alcoholic Fatty Liver Disease (NAFLD) and Non-Alcoholic Steato-Hepatitis (NASH), much like the adaptations resulting from exercise.

### Example 6

### METRNL Mediates Exercise-Induced Browning

It was then determined whether or not METRNL was required for the exercise-induced effects on the subcutaneous white fat. Injection of anti-METRNL neutralizing antibodies during 4 weeks of endurance training in HFD mice dramatically reduced the effect of endurance exercise on Ucp1 and Cidea gene expression, compared to injection of control antibody (FIG. 9). This indicated that endurance exercise-induced browning of subcutaneous fat that prevented diet-induced obesity is, at least in part, mediated by METRNL.

1. A method of treating metabolic disorders in a mammal comprising the step of administering to the mammal a meteorin-like protein (METRNL) or nucleic acid encoding METRNL.

2. The method of claim 1, wherein the METRNL is selected from human METRNL and functionally equivalent variants thereof which exhibit at least about 70% sequence similarity thereto.

3. The method of claim 1, wherein the METRNL is human METRNL.

4. The method of claim 1, wherein the nucleic acid encoding METRNL is selected from human nucleic acid encoding METRNL and functionally equivalent variants thereof which encode a functionally equivalent variant of METRNL that exhibits at least about 70% sequence similarity thereto.

5. The method of claim 1, wherein the METRNL or nucleic acid encoding METRNL induces at least one gene of the brown fat program in white adipose cells.

6. The method of claim 5, wherein the gene is at least one of the genes selected from the group UCP1, Prdm16 and Cidea.

7. The method of claim 1, wherein METRNL is administered in a dosage in the range of about 0.1 pg/kg to 4 ng/kg, or METRNL-encoding nucleic acid is administered in an amount that expresses about 0.1 pg/kg to 4 ng/kg METRNL.

8. The method of claim 1, wherein the METRNL or nucleic acid encoding METRNL is administered orally, subcutaneously, intravenously, intraperitoneally, intranasally, enterally, topically, sublingually, intramuscularly, intra-arterially, intramedullarily, intrathecally, ocularly, transdermally, vaginally, rectally or by inhalation.

9. The method of claim 1, wherein the METRNL or nucleic acid encoding METRNL is administered in conjunction with a second therapeutic agent.

10. The method of claim 9, wherein the second therapeutic agent is an agent useful to treat metabolic disorders.

11. The method of claim 10, wherein the second therapeutic agent is selected from a cytokine, insulin, betatrophin, adiponectin, leptin and a nutritional supplement.

12. The method of claim 1, wherein the metabolic disorder is a disorder resulting from obesity and obesity-associated co-morbidities.

13. The method of claim 1, wherein the metabolic disorder is selected from the group consisting of obesity, type 2 diabetes, insulin resistance, dysglycemia, Non-Alcoholic Steato-Hepatitis (NASH) and Non-Alcoholic Fatty Liver Disease (NAFLD).

14. A composition for the treatment of metabolic disorders comprising meteorin-like protein or nucleic acid encoding meteorin-like protein in combination with a pharmaceutically acceptable carrier.

15. The composition as defined in claim 14, comprising a second therapeutic agent.

16. The composition of claim 15, wherein the second therapeutic agent is an agent useful to treat metabolic disorders.

17. The composition of claim 16, wherein the second therapeutic agent is selected from a cytokine, insulin, betatrophin, adiponectin, leptin and a nutritional supplement.

18. Use of METRNL or nucleic acid encoding METRNL to treat metabolic disorders in a mammal.
